(19) United States
(12) Patent Application Publication
(10) Pub. No.: US 2004/0249350 A1
(43) Pub. Date: Dec. 9, 2004

(54) Device for fluid delivery system

(76) Inventor: Robert G. Rani, Vadnais Heights, MN (US)

Correspondence Address: NOVARTIS, CORPORATE INTELLECTUAL PROPERTY, ONE HEALTH PLAZA 430/2, EAST HANOVER, NJ, 07936-1080, US

(21) Appl. No.: 10/831,896

(22) Filed: Apr. 26, 2004

Related U.S. Application Data
(60) Provisional application No. 60/476,501, filed on Jun. 6, 2003.

(51) Int. Cl. A61M 5/00

(52) U.S. Cl. 604/251

(57) ABSTRACT

An apparatus and method for preventing the occurrence of free fluid in a fluid delivery system consists of a valve having a sealing element and elongate body disposed within the lumen of a flexible tube. The elongate body of the valve if further disposed within the inlet port of a connector. The perimeter of the sealing element and lumen of the flexible tube provide a tight seal preventing fluid flow when the flexible tube is in a relaxed state or when the longitudinal axis of the flexible tube is in alignment with the axis of the inlet port. The medical fluid is allowed to flow when the longitudinal axis of the flexible tube is not in alignment, e.g., when angularly stretched, with the axis of the inlet port.

### BACKGROUND OF THE INVENTION

The present invention relates to a device for preventing fluid free flow through a tube assembly of a fluid delivery system. More specifically, the present invention relates to an anti-free flow device, that prevents fluid free flow when the tube assembly is disengaged from a rotary pump, while allowing fluid flow the tube assembly is engaged with the pump.

1. Field of the Invention

Many individuals in hospitals or nursing homes cannot orally take nourishment or medication. These individuals, or medical patients, typically receive medical fluids containing the requisite nourishment and/or medication intravenously or-enterally via a tube assembly of a fluid delivery system. In such a fluid delivery system, gravity is used to feed the medical fluid from a container, e.g., a plastic pouch, through the tube assembly and into the patient. More recently, pumps, e.g., rotary peristaltic pumps, have been added to fluid delivery systems to regulate the rate that the medical fluid is infused into the patient.

Although these peristaltic pumps have allowed better control of the administration of the medical fluid, the use of the pumps have increased the risk that a medical patient is overmedicated or overfed. This risk arises whenever the tube assembly is disengaged from the pump, and the medical fluid is free to flow through the tube assembly under the force of gravity, in a situation known as fluid free flow.

To prevent the occurrence of free flow, many fluid delivery systems include valves or occluders which automatically block the passage of fluid whenever the tube assemblies are disengaged from the pumps. Although many of such valves and occluders are effective, they are also mechanically complicated and add to the overall cost of the tube assembly. Furthermore, many of these valves and occluders cannot be retrofitted to existing tube assemblies or easily incorporated into existing tube assembly designs. Other valves and occluders incorporate additional mechanical parts to the tube assembly, thus, making them more complex and difficult to use.

Thus, there is a need for a mechanically simple anti-free flow device that is compatible with existing tube assembly designs. An example of tube assembly that can be retrofitted with the anti-free flow device is the COMPAT® enteral delivery sets distributed by Novartis Nutrition Corporation (Minneapolis, Minn.).

### SUMMARY OF THE INVENTION

A primary object of the present invention to provide an apparatus and method for occluding the tube assemblies of feeding delivery sets to prevent free flow.

Another object of the present invention to provide an anti-free flow device which is simple to make, use and easily incorporated into existing tube assembly designs.

According to the present invention, there is provided a feeding delivery system that includes a container holding a medical fluid to be infused; a tube assembly containing a device for preventing free flow; and a pump, e.g., a rotary peristaltic pump, for pumping the medical fluid into the patient. The tube assembly includes components, such as a roller clamp, a drip chamber, an anti-free flow device, a connector and a spike, as well as medical grade tubing to connect these components in series. In addition to these components, other optional components can be incorporated into the tube assembly, e.g., a y-port fitting or roller clamp.

When the tube assembly is installed within the rotary peristaltic pump, the drip chamber and the connector are oriented, e.g., parallel to each other. The section of tubing connecting the drip chamber to the connector is wrapped, or stretched, around the rotor of the peristaltic pump.

When the tube assembly is installed within the rotary peristaltic pump and both the drip chamber and connector are each engaged with the pump, the section of tubing between the drip chamber and connector is stretched around the rotor of the peristaltic pump. The axes of the outlet port of the drip chamber and the inlet port of the connector are oriented, e.g., parallel to each other. The anti-free flow device consists, e.g., of a sealing element and an elongate body such that the elongate body is partially disposed within the inlet port of the connector. The longitudinal axis of the anti-free flow device resides in an non-parallel configuration with respect to the axis of the inlet port of connector. The longitudinal axis of the anti-free flow device and the axis of the inlet port of the connector intersect to form an angle, e.g., an acute example. The stretching of the tubing causes the tube to deform in the vicinity of the sealing element. This deformation allows the fluid to flow freely past the sealing element.

When the tube assembly is accidentally disengaged and no longer under tension, e.g., when the connector is no longer seated in the rotary peristaltic pump, the tubing relaxes to its original state prior to assemblage into the pump. The longitudinal axis of the anti-free flow device becomes collinear with the axis of the inlet port of the connector. A tight seal forms around the sealing element of the anti-free flow device thus preventing fluid from flow past the sealing element and onto the patient.

Numerous, other objects, features and advantages of the present invention will readily become apparent from the following detailed description, from the claims and from the accompanying drawings.

### BRIEF DESCRIPTION OF THE DRAWINGS

The accompanying drawings, which are incorporated in and constitute a part of the specification, illustrate an exemplary embodiment of the present invention.

FIG. 1 is a front elevational view of an exemplary embodiment of a fluid delivery system;

FIG. 2 is a front elevational view of an exemplary embodiment of a tube assembly for use with the fluid delivery system of FIG. 1;

FIG. 3 is a perspective view of an exemplary embodiment of an anti-free flow device in accordance with the present invention for use with the tube assembly of FIG. 2;

FIG. 4a is a front elevational view of the anti-free flow device of FIG. 3 within the tube assembly of FIG. 2 in a relaxed state;

FIG. 4b is an enlarged view of the section circled in FIG. 4a;

FIG. 5a is a front elevational view of the anti-free flow device of FIG. 3 within the tube assembly of FIG. 2 in a stretched state; and

FIG. 5b is an enlarged view of the section circled in FIG. 5a.

### DETAILED DESCRIPTION OF THE INVENTION

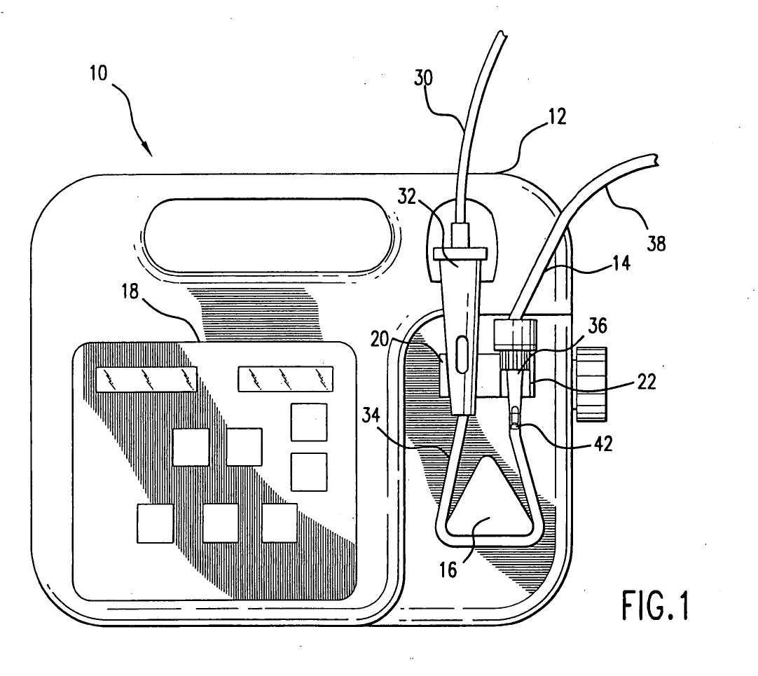

Referring to FIG. 1 a front elevational view of an exemplary embodiment of fluid delivery system 10 in accordance with the present invention is illustrated. The fluid delivery system 10 consists of pump 12 and tube assembly 14 assembled together in their intended operative manner.

The pump 12 can be any type of pump appropriate for controlling the flow of medical fluid through the tube assembly 14 and into a patient. For example, peristaltic pumps are commonly used, as known in the art, to accomplish such a task. The pump 12 selectively allows a metered amount of medical fluid to flow downstream or distally from the pump 12. Any type of peristaltic pump, for example a rotary peristaltic pump, can be used in conjunction with the anti-free flow device of the present invention. The pump, as depicted in FIG. 1., is a rotary peristaltic pump. The pump 12 has rotor 16 which engages the tube assembly 14 with rollers (not shown). Each revolution or partial revolution of the rotor 16 causes a specific amount of medical fluid to flow through the tube assembly 14. Also shown on the pump 12 is control panel 18 which allows a user to observe and adjust the rate of rotation for the rotor 16. A faster rate of rotation per period allows a larger quantity medical fluid to be administered to the patient. First bracket 20 and second bracket 22 are formed within the housing of the pump 12 and are located above the rotor 16. The first bracket 20 and second bracket 22, e.g., are used to engage various components of the tube assembly 14 which are discussed in further detail below.

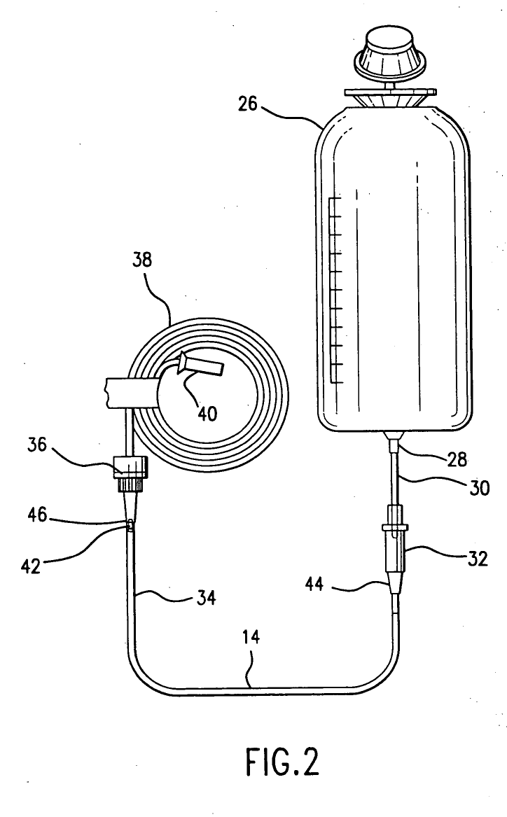

Referring to FIG. 2, a front elevational view of the tube assembly 14 in accordance with an exemplary embodiment of the present invention is depicted.

As used herein, the term “proximal” refers to the end of the tube assembly 14 closest to the supply container 26, and “distal” refers to the end of the tube assembly 14 furthest away from the supply container 26. Additionally, as used herein, the term “downstream” means in the direction of or nearer the patient. Likewise, as used herein, “upstream” means in the direction of or nearer the supply container 26.

The tube assembly 14 consists of a plurality of discrete, or integrally formed, components connected in series via sections of tubing. For example, at one end of the tube assembly 14 is supply container 26. The supply container 26 can be, e.g., a rigid bottle or a flexible pouch to hold the medical fluid that is to be administered to a patient enterally or parenterally. When in use, the supply container 26 is typically hung from a support (not shown) above the patient, e.g., five or six feet from off the floor.

At the bottom of the supply container 26 is outlet 28 which allows medical fluid to exit therefrom. Connected to the outlet 28 is first tube section 30. The first tube section 30 connects the supply container 26 to drip chamber 32. Second tube section 34 connects the drip chamber 32 to connector 36. Third tube section 38 links the connector 36 with patient connection 40. Aside from providing a physical means of attachment between the tube assembly 14 and pump 12, the drip chamber 32 also functions a feedback mechanism signaling to the pump 12 the rate the medical fluid is being administered.

For example, when the tube assembly 14 is for parenteral use, the patient connection 40 is a needle. When the tube assembly 14 is for enteral use, the patient connection 40 is a balloon catheter that connects to a stoma with a visceral organ, such as the stomach of the patient.

The tube sections 30, 34 and 38 are each made of a flexible, resilient material. The tube sections 30, 34 and 38 can be of identical or different materials. Examples of such materials include, but are not limited to, silicone, rubber, polyvinyl chloride, polyurethane, latex, neoprene or any other suitable medical grade material of the like. For example, the tube sections 30 and 38 can be made of polyvinyl chloride whereas the tube section 34 can be made of silicone.

Inserted within the lumen of the second tube section 34 is anti-free flow device 42 which is described in more detail below.

Referring back to FIG. 1 both the drip chamber 32 and the connector 36 are sized and shaped such that each can be attached to the first bracket 20 and the second bracket 22 of the housing of the pump 12 respectively. The section of tubing between the drip chamber 32 and the connector 36, the second tube section 34, is stretched around the rotor 16. The second tube section 34 connects outlet port 44 of the drip chamber 32 to inlet port 46 of the connector 36. The inlet port 46 serves as the entry to the bore (not shown) within the connector 36. The bore extends through the entire length of the connector 36 and provides a passage for the medical fluid to flow there through.

Referring now to FIG. 3, a perspective view of the anti-free flow device 42 is shown. The anti-free flow device 42 includes sealing element 52 and elongate body 54. The sealing element 52 and the elongate body 54 resemble, e.g., the head and stem of a nail.

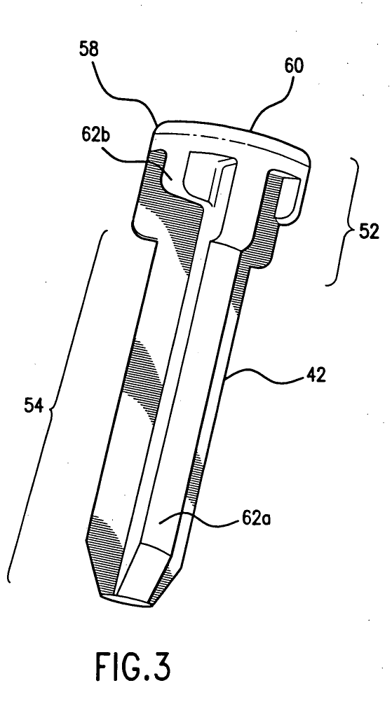

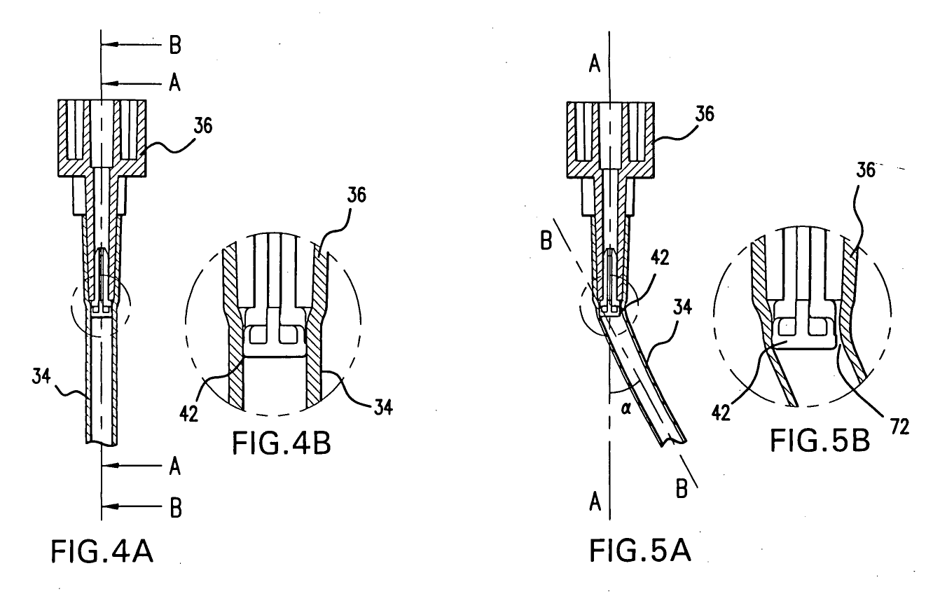

The sealing element 52, e.g., is disc-like, or circular, and has a diameter that is equal to or slightly greater than the inside diameter of the lumen of the tubing assembly 14 when the tubing assembly is relaxed or not placed under any tension. Perimeter 58 of the sealing element 52 and the inner wall of the lumen of the tubing assembly 14 create a fit, or a seal, preventing fluid from passing the sealing element 52 as shown in FIG. 4 (discussed in detail below). The sealing element 52 has top surface 60 which is oriented against the flow of the medical fluid within the tubing assembly 14. Top surface 60 can be planar or curved, e.g., convex or concave.

The elongate body 54 of the anti-free flow device 42 functions as a stem for the sealing element 52. The elongate body 54 can be rigid and does not bend whenever the second tube section 34 is pulled laterally or deformed. The shape of the elongate body 54 can be, e.g., tapered.

Located optionally along the length of the sealing element 52 are a plurality of grooves 62. The grooves 62, e.g., can be of any length and can run along the entire length of the anti-free flow device 42, including the exterior surface of the elongate body 54, shown as grooves 62a, or partially along the sealing element 54 shown as grooves 62b. The grooves 62 are oriented with respect to each other such that none of the grooves 62 are perpendicular to the adjacent groove. For example, in FIG. 3, each groove 62a and its adjacent groove 62b form an angle of 60°. If there were three grooves then the grooves would be spaced 120° apart. Any number of grooves in any angle of spacing can be used provided that no two adjacent grooves are perpendicular to each other.

Although the exemplary embodiment of the anti-free flow device 42 as shown in FIG. 3 resembles a nail, it is appreciated that one of ordinary skill in the art can develop alternative shapes for the anti-free flow device 42 in accordance with the spirit of the present invention. For example, the anti-free flow device can take on the shape of an egg, a disk, a ball and stem and a bullet.

FIGS. 4a and 4b show a front elevational view of the anti-free flow device 42 assembled within the lumen of the second tube section 34 of the tube assembly 14. FIG. 4a also shows the tube assembly 14 in a relaxed state which would be the state if the tube assembly 14 were not assembled with the pump or if the tube assembly 14 were accidentally disengaged. The anti-free flow device 42 is oriented such that the sealing element 52 is against the flow of the medical fluid within the tube assembly 14. The elongate body 54 is partially disposed within the bore of the connector 36 entering through the inlet port 46. “Partially disposed” means that some length of the elongate body 54 is within the bore of the connector. The elongate body 54 can be fixed or freely movable within the bore. For example, in an alternative part, the anti-free flow device 42 and the connector 36 can be integrally formed or be of a single construction.

The anti-free flow device 42 effectively prevents a free-flow condition whenever the tube assembly 14 is in a relaxed state which occurs whenever the second tube section 14 is not stretched or under tension. For example, a relaxed state occurs when the tube assembly 14 is inserted into the pump 12 or when the connector 36 become s disengaged from the pump 12 after the tube assembly 14 has already been connected to the pump 12.

When the tube assembly 14 is in a relaxed state, the perimeter of the sealing element 52 forms a seal with the lumen of the second tube section 34 to prevent the flow of medical fluid. In the relaxed state, the longitudinal axis, labeled B, of the second tube section 14 is axially aligned to axis, A, of the inlet port 46 of the connector 36. “Axial alignment” refers to the alignment between axes A and B, e.g., when the axes A and B are collinear or parallel.

When the tube assembly 14 is engaged in the pump 12, the second tube section 34 is in a stretched or flexed state, e.g., when stretched at an angle, and pulled laterally such that the axis B of the second tube section 34 is no longer in axial alignment with the axis A of the inlet port 45 of the connector 36. As shown in FIGS. 5a and 5b, the second tube section 34 is pulled towards the left of the connector 36. The axis A and axis B intersect to form angle α. When α is greater than 0°, e.g., 10°, 15°, 30°,45° or 60° the seal between the perimeter 58 of the sealing element 52 and the lumen of the second tube section 34 is temporarily breached to form passage 72 that allows the flow of medical fluid. Medical fluid flows through the lumen of the second tube section 34 into the passage 72 and along the grooves 62 of the anti-free flow device 42 and into the bore. Specifically, the passage 72 results from the reversible mechanical deformation of the second tube section 34 in the vicinity of the sealing element. Once the axes A and B are no longer in axial alignment, the sealing element 52 facilitates the flow of medical fluid by acting as a pivot point or lever that accentuates the passage 72.

During the engagement of the tube assembly 14 within the pump 12, the anti-free flow device 42 is stationary and remains in place regardless of what angle α is. In an alternative exemplary embodiment, the stretched state of the second tube segment 34 causes the elongate body 54 of the anti-free flow device 42 to pull out from the bore of the connector 36 such that the elongate body 54 is not as substantially disposed as it were prior to the stretching. Once again, the passage 72 is accentuated by allowing the anti-free flow device 42 to pull out of the connector 36. In this exemplary embodiment, the longitudinal axis of the anti-free flow device 42 is in axial alignment with the axis A when the second tube section 34 is in a relaxed state. The longitudinal axis of the anti-free flow device 42 is out of axial alignment with the axis A when the tube section is under a state of tension. The acute angle of intersection between the longitudinal axis of the anti-free flow device and the angle a can be the same or different in this exemplary alternative embodiment.

It is understood that while the present invention has been described in conjunction with the detailed description thereof that the foregoing description is intended to illustrate and not limit the scope of the invention, which is defined by the scope of the following claims. Other aspects, advantages and modifications are within the scope of the claims.

What is claimed:

1. A device for use in a fluid delivery system for use with a pump comprising:
a) a drip chamber;
b) a connector;
c) flexible tubing connecting said drip chamber and said connector allowing fluid communication therebetween, said flexible tubing having a lumen that allows a medical fluid to flow downstream from said drip chamber to said connector; and
d) a valve having a sealing element connected to an elongate body disposed within said lumen of flexible tubing such that said elongate body is partially disposed within said connector,
i) wherein said sealing element has a perimeter in contact with said lumen to form a seal preventing said medical fluid from flowing when said flexible tubing is in a relaxed state; and
ii) wherein a passage for said medical fluid to flow forms between said perimeter and said lumen when said flexible tubing is in a stretched state.

2. The device of claim 1, wherein said pump is a rotary peristaltic pump.

3. The device of claim 1, wherein said elongate body is entirely disposed within said connector.

4. The device of claim 1, wherein said elongate body is tapered.

5. The device of claim 1, wherein said sealing element is shaped like a disc.

6. The device of claim 5, wherein said sealing element has a plurality of grooves on said perimeter.

7. The device of claim 1, wherein said stretched state is an angularly stretched state.

8. A device for use in a fluid delivery system for use with a pump comprising:
a) a drip chamber;
b) a connector having an inlet port with an inlet port axis;
c) flexible tubing connecting said drip chamber and said connector allowing fluid communication therebetween, said flexible tubing having a longitudinal axis a lumen that allows a medical fluid to flow downstream from said drip chamber to said connector and
d) a valve having a sealing element connected to an elongate body disposed within said lumen of flexible tubing such that said elongate body is partially disposed within said connector through said inlet port and oriented along said inlet port axis,
i) wherein said sealing element has a perimeter in contact with said lumen to form a seal preventing said medical fluid from flowing when an angle formed by said longitudinal axis intersecting said inlet port axis is equal to 0°; and
ii) wherein a passage for said medical fluid to flow forms between said perimeter and said lumen when said angle is greater than 0°.

9. The device of claim 8, wherein said pump is a rotary peristaltic pump.

10. The device of claim 8, wherein said elongate body is entirely disposed within said connector.

11. The device of claim 8, wherein said elongate body is tapered.

12. The device of claim 8, wherein said sealing element is shaped like a disc.

13. The device of claim 12, wherein said sealing element has a plurality of grooves on said perimeter.

14. A method of preventing free-flow in a flexible tube of a fluid delivery set having a drip chamber connected to a connector by the flexible tube comprising the steps of:
a) positioning a valve having a sealing element and an elongate body within said fluid delivery set wherein a perimeter of said sealing element forms a seal with a lumen of said flexible tube and said elongate body is disposed within an inlet port of said connector;
b) allowing a medical fluid to flow through a passage created between said perimeter and said lumen when said flexible tube is in a stretched state; and
c) not allowing said medical fluid to flow through said flexible tube when said flexible tube is in a relaxed state.

15. The method of claim 14, wherein said fluid delivery set is engaged in a rotary peristaltic pump having a rotor.

16. The method of claim 15, wherein said stretched state results from said flexible tube being wrapped around said rotor.

17. The method of claim 16, wherein said sealing element is shaped like a disc.

18. The method of claim 16, wherein said sealing element is tapered.

19. A valve assembly attached to a flexible tubing having an inner wall forming a lumen, said valve assembly comprising a device, said device having a peripheral surface that conforms to and contacts with said inner wall, thereby forming a fluid seal to close said lumen, wherein said peripheral surface minimally contacts said inner wall such that when said flexible tubing is angularly flexed, said fluid seal is breached.
